(19) United States
(12) Patent Application Publication
(10) Pub. No.: US 2008/0300526 A1
(43) Pub. Date: Dec. 4, 2008

(54) GUAYULE RUBBER AND RESIN WET-STICK BIOADHESIVES

(75) Inventor: Ronald W. Gumbs, East Brunswick, NJ (US)

Correspondence Address: JENNINGS, STROUSS & SALMON, P.L.C., 201 E. WASHINGTON ST., 11TH FLOOR, PHOENIX, AZ, 85004, US

(73) Assignee: Yulex Corporation

(21) Appl. No.: 11/757,239

(22) Filed: Jun. 1, 2007

(51) Int. Cl. A61F 13/02

(52) U.S. Cl. 602/54

(57) ABSTRACT

A bioadhesive for bonding to wet skin is disclosed, including a novel non-Hevea-based resin, which serves as a tackifier for the rubber, possessing strong wet adhesion to human skin and the remarkable property of bonding to it underwater, and a novel non-Hevea rubber that provides cohesive strength to the adhesive. Pressure-sensitive adhesive tapes are used in many applications where there is a need to adhere to skin, for example, medical tapes, wound or surgical dressings, athletic tapes, surgical drapes, or tapes or tabs used in adhering medical devices such as sensors, electrodes, ostomy appliances, and so on.

### FIELD OF THE INVENTION

This invention relates in general to pressure-sensitive skin adhesives, and more specifically to adhesives based on natural rubber and resin derived from any of a large number of plant species bearing rubber and rubber-like hydrocarbons, including, but not limited to, guayule (Parthenium argentatum gray).

### BACKGROUND OF THE INVENTION

Since the dawn of civilization, various glues have been used in wound dressing and in surgical repair. Ancient Egyptians used strips of linen coated with natural glues, such as flour, honey and other sticky substances, for application across gaping flesh wounds. The dry adhesion of these materials is at least 20 g/2.5 cm (0.08 N/cm).

Egyptians discovered over 4,000 years ago that bonding to skin is relatively easy without the knowledge of the functional groups on the collagen molecule. Included among these groups are the following: carboxylic groups; amino groups; guanidines; phenolics; amino alcohols; and sulfhydrils (thiols). The word collagen means glue producer and the oldest glue known to man, and carbon dated as more than 8,000 years old, is collagen.

During the last century, surgical tapes typically contained an adhesive layer of natural rubber from the Brazilian rubber tree and zinc oxide. Various additives have been used to improve adhesion and reduce irritation. Adverse skin reactions to this type of tape have been experienced by most patients for many reasons, including the following: allergic reactions, and trauma caused by the barrier imposed on the passage of fluids through the skin.

Medical-grade acrylic adhesives are universally employed as replacements for natural rubber. One spin-off of this technology is the transdermal delivery of pharmaceuticals, where a patch is attached to the skin or mucous membrane to enable the controlled release of the drug to the body. In addition to wound healing, the bioadhesives are used in dressings for various catheters (peripheral arterial, central venous and other sites.)

Direct application of some bioadhesives to the wound is the most sophisticated application of medical grade adhesives. For instance, US Army scientists discovered that anhydrous polymers, such as polyacrylic acids, that are covalently cross-linked to form unique macromolecules known as carbomers, which, when hydrated, present superficial carboxyl groups for strong bioadhesion to wet tissues. The macromolecules absorb water rapidly enough to concentrate blood clotting factors but slowly enough to remain bioadhesive until clotting is complete.

Polyacrylic acid is the prototypical bioadhesive because it bonds to all areas of wet skin, including mucous tissue. In view of this, virtually all synthetic adhesive replacements for natural rubber adhesives contain a significant concentration of carboxylic acid groups to facilitate bonding to wet skin.

There are two main classes of bioadhesives disclosed in the patent literature and these relate to their solubility in water. Water-soluble, pressure-sensitive skin adhesives include copolymers of salts of acrylic acid. Insoluble skin adhesives contain polymers that do not dissolve in water, including both thermoplastic and cross-linked materials. The cross-linked polymers include hydrogels, which consist of a gel with various amounts of water. Hydrogels serve as a plasticizer and a vehicle for transdermal delivery of biocides and other medication to aid in wound healing. These exhibit the required wet adhesion and can be applied directly to open wounds. Skin adhesive hydrogels based on polyvinyl pyrrolidone and hydrogels based on cross-linked acrylic polymers with excipient groups such as acrylic acid units are known in the art.

Bioadhesives also have been described based on cross-linked methacrylic hydrogels in mixtures with pressure-sensitive methacrylic ester polymers specially formulated for wet-stick adhesion. The presence of acidic monomers, e.g., 8-12% acrylic acid in the film-forming component, combined with a hydrophilic plasticizer in the hydrogel is critical, while the hydrogel absorbs water on wet skin. The addition of absorbent particulate material, capable of absorbing at least 50 times its weight in water, to a fibrous web represents another approach to achieving wet-stick adhesion in articles. Test protocols using human volunteers are described and instructive; initial wet-skin adhesion of at least 0.08 N/cm is reported and 20 μL of water was used to wet an area of skin 2.5 cm wide and 7.6 cm long.

One approach to achieving satisfactory wet adhesion is the use of pattern-coated adhesives where, for example, a discontinuous adhesive coating on a backing is used to permit the skin to breath in the areas of the backing not coated with adhesive. These involve intermittent coating of adhesives onto different backings. A release-coated calendar roll similar to gravure printing is employed, as well as screen printing. Also, articles possessing good wet-skin adhesion comprise a porous backing comprised of non-wettable fibers and a discontinuously-coated adhesive is used. The backing absorbs less than 4% by weight water, thereby enabling water on wet skin to pass through the entire article.

Another approach that is widely used involves increasing the hydrophilic character of methacrylate polymers through copolymerization with hydrophilic acidic comonomers, such as acrylic acid, methacrylic acid, beta-carboxyethyl acrylate, itaconic acid, sulfoethyl acrylate, and the like. Incorporation of these monomers in minor amounts (1-15%) lowers tack. At higher levels of acid, there is a dramatic loss of tack and the copolymer becomes highly hydrophilic. When exposed to water, the moisture helps to transform these highly acidic, low-tack compositions into tacky materials that are suitable as wet-stick adhesives used in many medical applications. When the water is allowed to evaporate, however, these bioadhesives lose their pressure-sensitive tack. Even though the adhesion is satisfactory in limited applications, there is still a need for articles with good initial wet adhesion.

Surgical pressure-sensitive adhesive compositions having improved long-term skin adhesion characteristics comprising natural rubber, and a cross-linked hydrophilic random interpolymer (hydrogel) have also been described. The composition of the rubber-based pressure-sensitive adhesive, reproduced in Table 1 below, exhibits a long-term skin adhesion value of 80.

TABLE 1
Composition of Surgical Tape based on Natural
Rubber known in the art.

| | Parts by |
|---|---|
| Ingredient | weight |
| Pale crepe (Hevea) rubber | 31.3 |
| Tackifier (mixture of rosin acids) | 28.2 |
| Aluminum hydrate | 12.1 |
| Zinc oxide | 9.7 |
| Lanolin | 9.6 |
| Corn starch | 4.7 |
| Titanium dioxide | 2.3 |
| Water | 0.7 |
| Dibutyldithio zinc carbamate | 1.4 |
| TOTAL | 100.0 |

Long term adhesion increases to 90 with addition of 9% of the cross-linked hydrophilic interpolymer, the composition of which is given in Table 2.

TABLE 2
Composition of Cross-Linked Hydrophilic
Interpolymer known in the art.

| Ingredients | Parts by weight |
|---|---|
| 2-Hydroxy-3-methacryloxypropyl | 45 |
| trimethyl ammonium chloride | |
| Acrylamide | 45 |
| Acrylic acid | 10 |
| N,N′-methylene bisacrylamide | 0.05 |

Here, “long-term skin adhesion” refers to the degree of adherence of the pressure-sensitive adhesive mass to the human skin at 24 hours after application thereof. The long-term skin adhesion of a particular adhesive mass may be determined in accordance with the following test: 1×3 inches tapes comprising a suitable backing material coated with the adhesive to be tested are placed on the upper arm of a number of human subjects and left there for 24 hours, during which time the subjects pursue their normal activities. At the end of the test period the tapes are checked for skin adherence and rated on a scale of from 0 to 100. Where essentially no separation of tape from the skin, such as lifting from the corners or other partial removal, has occurred, the long term skin adhesion is given a rating of 100 (perfect adhesion). Where the tape has completely separated from the skin of the test subject, the long term skin adhesion is rated as 0 (complete failure). Intermediate degrees of adhesion are assigned values between 0 and 100 with higher values being indicative of better adhesion characteristics. Each adhesive-coated tape is tested on a number of subjects (usually 24) and the individual test results are averaged to give the final score.

One major concern with these skin adhesives is the need for sufficiently high levels of adhesion to wet skin. Conventional pressure sensitive adhesives based on the underlying technology (e.g., acrylics) typically adhere to various dry surfaces with peel adhesive strengths significantly higher than 2,000 g/2.5 cm. Commercially available bioadhesives do not bond to wet fingers. Further, they lose their adhesive properties upon immersion in water because of their hydrophilic nature. Water is the substance that poses the greatest problems in terms of environmental stability for bioadhesive joints, because it can degrade the properties of the bulk adhesive, particularly at the interface. If a dressing ceases to stick to the skin, it is no longer effective and therefore it must be changed

Further, present bioadhesives cannot achieve a long-term skin adhesion close to 100, with the closest known being 90. Further, no rubber-based bioadhesives have been shown to have a long term skin adhesion over 80. Additionally, the rubber-based bioadhesives known in the art are comprised of Hevea rubber. This is problematic for several reasons.

The vast majority of Hevea-derived natural rubber is grown from a limited number of cultivars in Indonesia, Malaysia and Thailand, using labor-intensive harvesting practices. The rubber and products made from Hevea are expensive to import to other parts of the world, including the United States, and supply chains can limit availability of materials. Furthermore, because of the restricted growing area and genetic similarity of these crops, plant blight, disease, or natural disaster have the potential to wipe out the bulk of the world's production in a short time.

Second, particularly in the medical and patient care areas, an estimated 20 million Americans have allergies to proteins found in the Southeast Asian Hevea-derived natural rubber crop. Like many other plants, Hevea produces proteins for structural support and for defense-related purposes in response to environmental conditions. However, there are at least 62 known Hevea antigens involved in Type I latex allergy, and more than a dozen of these Hevea-derived latex proteins are common human allergens, including: Hev b1, and Hev b3 implicated in rubber biosynthesis, defense related proteins Hev b2, Hev b4, Hev b6.01, Hev b6.02, Hev b6.03, Hev b7.01, Hev b7.02, Hev b11, and Hev b12, and other proteins such as Hev b5, Hev b8, Hev b9, and Hev b10.

An allergic response to Hevea begins when a latex-allergic individual is exposed to these proteins, triggering immunoglobulin E (“IgE”) antibody production. The IgE antibodies cause a variety of responses, depending on the severity of the allergy. Typically, latex allergies are limited to skin inflammation, but serious reactions, and even death, may occur in some individuals.

Therefore, a need exists for a non-Hevea natural rubber-based, wet-stick bioadhesive capable of achieving a superior long-term adhesion rating.

### BRIEF DESCRIPTION OF THE DRAWINGS

FIG. 1 illustrates one embodiment of the presently-disclosed bioadhesive.

FIG. 2 illustrates an alternate embodiment of the presently-disclosed adhesive structure, namely a double-sided bioadhesive structure.

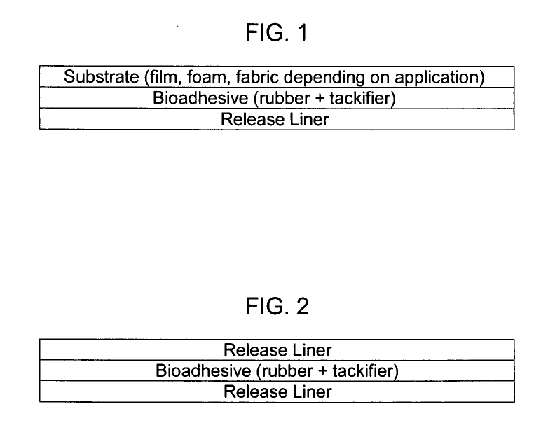

### DETAILED DESCRIPTION OF THE INVENTION

A wet-skin adhesive comprised of guayule or other non-Hevea rubber and a tackifier, which consists of a mixture of guayule (Parthenium argentatum) or other non-Hevea resin and a polyterpene, is disclosed. The hypoallergenic rubber preferably extracted from the defoliated plant as latex using a water extraction process bonds to dry skin, but not to wet human skin. Adhesion to wet human skin increases with concentration of tackifier, but the optimal level is the minimum concentration necessary to provide tack so that self-supported or transfer adhesive films can be fabricated. Advantageously, the wet-adhesion can be regulated by adjusting the ratio of rubber to tackifier, and achieves the peel adhesion required for bioadhesives of 20 grams/cm; rubber-based adhesives for non-medical applications typically exhibit peel adhesion to many substrates well above 2,000 grams/cm.

Its primary advantages over existing medical adhesives are superior wet-finger adhesion, water resistance, and flexibility. Its adhesive characteristics can be varied by controlling the ratio of rubber to tackifier. Its advantage over Hevea-rubber bioadhesives is based on its hypoallergenic character, attributed to a significantly lower concentration of proteins, and a complete absence of protein epitopes that can cross-react with Type I latex allergy. Commercially available bioadhesives lose their adhesive properties after immersion in water because they depend on their hydrophilic nature for wet-adhesion. Guayule rubber and other non-Hevea rubber bioadhesives of the present invention do not lose their adhesive properties because they are hydrophobic. Adhesion to wet skin is easily achieved with the addition of a few more parts of either resin or polyterpene, but the latter is preferred.

According to the present disclosure, examples of non-Hevea natural rubber and resin sources include, but are not limited to, guayule (Parthenium argentatum), gopher plant (Euphorbia lathyris), mariola (Parthenium incanum), rabbitbrush (Chrysothamnus nauseosus), milkweeds (Asclepias sp.), goldenrods (Solidago sp.), pale Indian plantain (Cacalia atripilcifolia), rubber vine (Crypstogeia grandiflora), Russian dandelion (Taraxacum sp. and Scorzonera sp.), mountain mint (Pycnanthemum incanum), American germander (Teucreum canadense) and tall bellflower (Campanula america). All of these non-Hevea natural rubber sources are capable of being evaluated according to the present disclosure to determine suitability for use in the disclosed non-Hevea natural rubber-based bioadhesives.

In particular, guayule (Parthenium argentatum), a desert plant native to the southwestern United States and northern Mexico, produces rubber essentially identical, or of improved quality, when compared with Hevea. Thus, the terms non-Hevea natural rubber and guayule rubber are used interchangeably in the present disclosure, as well as the terms non-Hevea-based resin and guayule-based resin. Additionally, processed guayule rubber and resins have no proteins that related to the allergenic properties of Hevea.

As used in this disclosure, “pressure-sensitive adhesive” is a viscoelastic material that displays aggressive tackiness and adheres to many surfaces after the application of light pressure. Further, “wet-stick adhesive” refers to a material that exhibits pressure-sensitive adhesion when adhered to at least a wet surface, preferably and particularly skin. Finally, “resin” refers to a mixture of low-molecular-weight rubber and various terpenoids, triglycerides of fatty acids extracted with acetone from guayule or guayule bagasse.

The present disclosure further provides for certain mixtures of guayule rubber and resin or polyterpenes that possess strong adhesion to wet human skin and the exceptional property of bonding to it and other surfaces underwater. Briefly, in one aspect of the invention, a wet-stick adhesive comprising of a mixture of guayule rubber and tackifier is provided, wherein the pressure-sensitive adhesive adheres to wet skin. Advantageously, the bioadhesives in accordance with the present disclosure adhere to wet human skin and do not lose their adhesive qualities under water.

According to the present disclosure, the concentration of rubber component present in the adhesive is between 75 percent by weight (wt. %) to about 95 wt. % and the concentration of resin or polyterpenes is about 5 wt. % to 25 wt. %, based on the total weight of rubber and tackifier. Further, the wet-stick, pressure-sensitive adhesive may contain organic solvents, reactive diluents and initiators or mixtures thereof. The wet-stick pressure-sensitive adhesive may be water-based and applied by adding the tackifier to the latex compound or dispersion used to make gloves and other articles. The wet-stick, pressure-sensitive adhesive can be cross-linked in order to improve cohesive strength and strippability. A further aspect of the invention provides a method of making a wet-stick, pressure-sensitive, the method comprising combining rubber and tackifier in a liquid medium, after extraction, separation and purification.

As noted above, certain mixtures of guayule rubber and resin or polyterpenes possess strong adhesion to human skin and the exceptional property of bonding to it and other surfaces underwater. However, current pressure-sensitive adhesive films marketed for adhesion to skin do not adhere to a wet finger and lose their adhesive properties after immersion in water. Generally, rubber is more cohesive than it is adhesive to human skin and it is removable from skin without leaving a noticeable residue, if cured. It does not adhere to wet skin. Resin and polyterpenes on the other hand are more adhesive than cohesive to wet and dry human skin; they can only be removed with considerable difficulty, typically with mild abrasives and organic solvents.

Hevea, guayule and other non-Hevea rubber-producing plants identified above are bioadhesive factories because they produce natural rubber, resins, terpenoids and oleic acid triglycerides. Guayule and certain other non-Hevea plants with higher concentrations of resin and lower concentrations of proteins are superior and more efficient bioadhesive plants. The reasons for this conclusion are based on the physical and chemical nature of both the resin and the rubber.

This disclosure is primarily focused on the wet-stick adhesive, rather than on articles or objects coated with the adhesive. Suffice to say, fabrication of the guayule bioadhesive would be similar in configuration to the current products, except that instead of a medical-grade acrylic adhesive, a composition comprising of natural rubber and its tackifier (resin) is employed in the prototypical laminate structure, depicted schematically.

The bioadhesives of the present disclosure are prepared according to the following.

Extraction of rubber. Guayule plants are pulverized by a hammer mill or other similar grinding apparati and the rubber is first isolated using water as described in two patents (K. Cornish, “Hypoallergenic natural rubber products from Parthenium argentatum (gray) and other non-Hevea brasiliensis species,” U.S. Pat. No. 5,717,050, Feb. 10, 1998; K. Cornish, “Hypoallergenic natural rubber products from Parthenium argentatum (gray) and other non-Hevea brasiliensis species,” U.S. Pat. No. 5,580,942, Dec. 3, 1996, or with a simultaneous solvent extraction as taught and reviewed by Schloman in U.S. Pat. No. 6,054,525 with an acetone/pentane extract to yield a swollen rubber miscella. Rubber and resin can also be extracted using supercritical carbon dioxide, supercritical carbon dioxide with cosolvent, or accelerated solvent extraction methods. See U.S. Pat. App. 2006/0106183, May 18, 2006.

Purification of rubber. The swollen rubber mass from solvent processes contains residual solvents and resin which must be removed to obtain pure rubber. This can be accomplished by adding sufficient acetone to precipitate rubber and extract all of the resin. Evaporation of the volatiles yields the two separate streams. Even though the chemical structure (cis-1,4-polyisoprene) of guayule rubber is identical to that of Hevea rubber, significant differences exist that illustrate the superior adhesive properties of the present invention. Alternatively, in the superciritical processes, the resin and cosolvent are separated from the rubber phase using increasing levels of supercritical carbon dioxide.

Guayule rubber comprising one to two percent (1-2%) resin has excellent tack and adheres to dry skin. The bonding mechanisms in guayule rubber bioadhesives are more diverse when the rubber is blended with guayule resin. Because it is hydrophobic, the guayule rubber bioadhesive has very good water and moisture resistance. The flexibility of the bioadhesive is very high. It bonds to a wide range of substrates, above and below water. The presence of the resin increases the curing opportunities, while demonstrating increased versatility. It is completely miscible with hydrocarbon solvents and free of gel. It is soluble in some nonvolatile acrylates.

Test Protocols. Evaluation of adhesive bonding to wet skin is somewhat problematic because of the wide variations in composition, topography, and the presence/absence of different body fluids. Guayule resin and other materials with wet-stick properties bond aggressively to wet human skin and this inventor assumes that the peel adhesion can be controlled with the use of very thin films that are breathable and easy to remove without causing skin trauma.

“Wet-finger adhesion” refers to the degree of adherence of the pressure-sensitive adhesive mass between a wet thumb and wet index finger immediately after application. It may be determined by immersing the fingers in water for approximately one minute and then coating one finger with the adhesive. Both fingers are then brought into contact to simulate bonding and the degree of difficulty in removing the adhesive is noted. The type of failure is recorded as either cohesive or adhesive, and the amount of residue remaining after stripping is weighed or estimated. Free-standing films, if available, also can be tested using this procedure, as well as tapes comprising a suitable backing material coated with the adhesive. At the end of the test period, the fingers are checked for skin adherence and rated on a scale of 0 (no adhesion) to 10 (perfect adhesion). Adhesion can also be measured while the adhesive is immersed in water.

### EXAMPLES

The embodiments of the present disclosure are further illustrated by the following examples that are not intended to limit its scope. In the examples, all parts, ratios and percentages are by weight unless otherwise indicated.

### Example 1

### Extraction

Residual guayule bagasse after water extraction of latex was simultaneously extracted with an acetone/pentane azeotrope as described by Schloman, Jr. The product of this example was a rubber-resin miscella, which after evaporation of solvent, contained about 60% rubber and 40% resin.

### Example 2

### Separation

The product from Example 1 was poured into a large excess of acetone to precipitate the rubber with stirring; rubber and resin were recovered after evaporation of the solvent. Extraction with refluxing acetone using the Soxhlet procedure indicated that the rubber contained 1.6% either polyterpenes or guayule resin. It is important to note that the latter contains low molecular weight rubber or polyterpenes; rubber is itself a polyterpene.

### Example 3

### Preparation of Adhesives

Coagulated latex was guillotined and stirred in 1:1 mixture of xylene and tetrahydrofuran at room temperature to extract soluble rubber. After removal of the insoluble materials, the rubber was isolated. Cements were prepared by adding 25 parts of rubber to 75 parts toluene in a glass container. After the mixture was magnetically stirred at room temperature for 8 hours, a miscible solution free of insoluble material was formed and used to prepare the compositions in Examples 4-14 listed in Table 3.

TABLE 3
Preparation of Bioadhesives

| Example | | | | | Dry | Wet |
|---|---|---|---|---|---|---|
| # | Rubber | Resin | Poly (α-pinene) | Poly (β-pinene) | Adhesion | Adhesion |
| 4 | 100 | 0 | | | 100 | 0 |
| 5 | 94 | 6 | | | 100 | 0 |
| 6 | 89 | 11 | | | 100 | 50 |
| 7 | 85 | 15 | | | 100 | 100 |
| 8 | 80 | 20 | | | 100 | 100 |
| 9 | 77 | 23 | | | 100 | 100 |
| 10 | 73 | 27 | | | 100 | 100 |
| 11 | 85 | 0 | 15 | | 100 | 100 |
| 12 | 85 | 0 | | 15 | 100 | 100 |
| 13 | 70 | 0 | 15 | 15 | 100 | 100 |
| 14 | 70 | 15 | 0 | 15 | 100 | 100 |

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of a preferred embodiment and best mode of the invention known to the applicant at this time of filing the application has been presented and is intended for the purposes of illustration and description. It is not intended to be exhaustive or limit the invention to the precise form disclosed and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out this invention, but that the invention will include all embodiments falling within the scope of the appended claims.

What is claimed is:

1. A wet-stick pressure-sensitive adhesive comprising: non-Hevea rubber and a tackifier, wherein the adhesive is capable of adhering to wet skin.

2. The wet-stick pressure-sensitive adhesive of claim 1, wherein the tackifier comprises a non-Hevea resin.

3. The wet-stick pressure-sensitive adhesive of claim 1, wherein the non-Hevea rubber is guayule rubber.

4. The wet-stick pressure-sensitive adhesive of claim 2, wherein the non-Hevea resin is guayule resin.

5. The wet-stick pressure-sensitive adhesive of claim 1, wherein the tackifier comprises a polyterpene.

6. The wet-stick pressure-sensitive adhesive of claim 5, wherein the polyterpene is poly α-pinene.

7. The wet-stick pressure-sensitive adhesive of claim 5, wherein the polyterpene is poly β-pinene.

8. The wet-stick pressure-sensitive adhesive of claim 2, wherein the tackifier further comprises a polyterpene.

9. The wet-stick pressure-sensitive adhesive of claim 1, wherein the concentration of non-Hevea rubber in the adhesive is present in an amount of in the approximate range of 75 percent to 95 percent, based on the total weight of dry solids.

10. The wet-stick pressure-sensitive adhesive of claim 1, wherein the concentration of the tackifier in the adhesive is present in an amount in the approximate range of 5 percent to 25 percent, based on the total weight of dry solids.

11. The wet-stick pressure-sensitive adhesive of claim 1, further comprising organic solvents.

12. The wet-stick pressure-sensitive adhesive of claim 1, further comprising reactive diluents.

13. The wet-stick pressure-sensitive adhesive of claim 1, further comprising initiators.

14. The wet-stick pressure-sensitive adhesive of claim 1, further comprising a mixture of organic solvents, reactive diluents, and initiators.

15. The wet-stick pressure-sensitive adhesive of claim 1, wherein the adhesive is cross-linked, the cross-linking capable of increasing cohesive strength.

16. The wet-stick pressure-sensitive adhesive of claim 1, wherein the cross-linking is further capable of increasing strippability.

17. A water-based wet-stick pressure-sensitive adhesive comprising: guayule rubber and tackifier, wherein the adhesive is prepared by adding the tackifier directly to the latex formulation used to make gloves and other articles.

18. A method of making a wet-stick pressure-sensitive adhesive, comprising combining non-Hevea rubber and tackifier in a liquid medium.

19. The method of claim 18, wherein the liquid medium is water.

20. The method of claim 18, wherein the tackifier is non-Hevea resin.
